(19) United States
(12) Patent Application Publication
(10) Pub. No.: US 2007/0134307 A1
(43) Pub. Date: Jun. 14, 2007

(54) Antimicrobial composite and method of manufacture and use

(76) Inventors: T. Danny Xiao, Willington, CT (US); Jinxiang Dai, Mansfield, CT (US); Junfeng Zhou, Mansfield Ctr., CT (US); Meidong Wang, Willington, CT (US); Michael S. Gray, Willingboro, NJ (US); Gregory J. Robb, Lawrenceville, NJ (US); Barry E. Constantine, Island Heights, NJ (US); David E. Reisner, Bristol, CT (US); Matthew L. Harriton, New York, NY (US)

Correspondence Address: CANTOR COLBURN, LLP, 55 GRIFFIN ROAD SOUTH, BLOOMFIELD, CT, 06002, US

(21) Appl. No.: 11/474,817

(22) Filed: Jun. 26, 2006

Related U.S. Application Data
(60) Provisional application No. 60/693,769, filed on Jun. 24, 2005.

(51) Int. Cl. A61F 15/00; A61L 15/16; A61F 13/00

(52) U.S. Cl. 424/447; 602/41

(57) ABSTRACT

An antimicrobial composite is provided, comprising a catalyst capable of promoting oxidation of organic molecules incorporated in a carrier, for example a hydrophilic or water-based material, the catalyst configured such that it will not discolor surrounding material under the influence of oxidative conditions

### CROSS REFERENCE TO RELATED APPLICATION

This application claims the benefit of U.S. Provisional Patent Application Ser. No. 60/693,769 filed Jun. 24, 2005, which is incorporated herein by reference in its entirety.

### BACKGROUND

The present disclosure is directed generally to incorporation of a catalyst capable of promoting oxidation of organic molecules, such as bacteria, into a carrier, for example a hydrophilic or water based material, such as a hydrogel, the catalyst (e.g., silver flakes or other silver particle) sized or configured to prevent discoloration of such material or surrounding material upon oxidation at the catalyst.

A wide variety of disposable absorbent articles designed not only to be efficient in the absorption of body fluids (e.g. urine, blood, menses, and the like), but also to be sanitary and comfortable in-use, are known in the literature. Disposable absorbent products of this type generally comprise a fluid-permeable topsheet material, an absorbent core, and a fluid-impermeable backsheet material. Various shapes, sizes and thicknesses of such articles have been explored in an attempt to make their use more comfortable and convenient.

More recently, research has been focused on the removal of foul odors and the prevention of skin diseases such as dermatitis, rash and redness caused by wearing a disposable absorbent article for a relatively long time. Many body fluids have an unpleasant odor, or develop such odors when in contact with air and/or bacteria for prolonged periods. Additionally, urine and/or other exudates absorbed into the absorbent article are converted to ammonia by urease produced by skin-flora (i.e., a group of normal microorganisms on the skin). This ammonia, in turn, causes dermatitis, rash, and/or other forms of skin irritation. Such disease of the skin in infants can be a serious medical matter, which, in extreme cases, can result in death.

Additionally, superficial topical infections are typically a consequence of a primary disease source such as chronic urinary incontinence, or are directly related to a contagious nosocomial or endemic source. Prolonged moist or wet skin conditions often lead to maceration and other changes in skin integrity, which provide the opportunity for normally saprophytic bacteria and fungi to invade the site and establish an infection.

Wounds that are heavily contaminated by microorganisms, but not clinically infected, are often characterized by a prolonged period of inflammation, as well as a delay in wound repair and healing. Microorganisms that contaminate wounds have been implicated as an important factor in the retardation of wound healing by interfering with leucocyte phagocytosis, and by the depletion of nutrients and oxygen required for normal tissue granulation.

A large number of dressings, bandages, and topic medicaments are available for the treatment of these and other types of wounds. These products fall into two categories, passive and active. Passive wound dressings are dressing which serve only to provide mechanical protection and a barrier to infection. The dressings themselves do not supply any composition that enables or facilitates the healing process of the wound. Examples of passive dressings include gauze and adhesive bandages. Active dressings are dressings that supply some biologically active compound to the site of a wound. One type of active dressing is a dressing or wrapping that delivers or has been impregnated with antimicrobials.

Silver, delivered as an antimicrobial agent, either as a salt in an emulsion (an example is Silvadene® Cream) or as a solubilized form of silver in a nanocrystalline silver dressing (an example is Acticoat®), is deposited in the repair tissue and discolors when influenced by oxidative action of water and light. As the regenerative tissue containing the silver matures, the silver remains and is easily recognized by its discoloration when compared to the pigmentation of the surrounding tissue. Indeed, discoloration of both the wound and the dressing is a pervasive problem with regard to wound dressings or the like incorporating such silver.

Accordingly, what is needed is a material (e.g., a wound or other dressing having antimicrobial qualities, anti-odor patches, inserts or cushioning, antibacterial adhesives, topical skincare, cleaning agents, or the like) that enjoys the benefits associated with incorporation of silver (or other catalyst capable of promoting oxidation of organic molecules) as an active material, but that does not suffer disadvantages associated with discoloration of surrounding material or tissue.

### BRIEF SUMMARY

The above-described disadvantages are overcome and alleviated by the presently described antimicrobial composite, comprising a catalyst capable of promoting oxidation of organic molecules incorporated in a carrier, for example a hydrophilic or water-based material, the catalyst configured such that it will not discolor surrounding material under the influence of oxidative conditions. In one embodiment, the catalyst has a size sufficient such that oxidation occurs on the silver within the hydrophilic or water-based material and such that the catalyst (e.g., a silver flake or particle) does not leach out from the carrier. In another embodiment, such catalyst has an irregular geometry, such that catalyst oxidation occurs on the surface of the catalyst and such that the catalyst does not leach out from the carrier.

In another embodiment, the hydrophilic or water-based material is a hydrogel having antimicrobial activity. The hydrogel includes a hydrogel-forming polymer and an antimicrobial agent comprising a silver- flake or particle. The hydrogel is produced from a hydrogel-forming polymer, such as a hydrophilic polymer, with water and crosslinks the polymer and water using an energy source. The method does not need any chemical additive to affect the crosslinking, unlike prior art methods of forming hydrogels. The antimicrobial agent may be mixed with the hydrogel-forming polymer and water prior to crosslinking. Alternatively, the antimicrobial agent may be applied to a substrate onto which the hydrogel is placed such that the antimicrobial agent migrates into the hydrogel.

The above described and other features are exemplified by the following figures and detailed description.

### BRIEF DESCRIPTION OF THE DRAWINGS

Referring now to the figures, which are exemplary embodiments and wherein like elements are numbered alike:

FIG. 1 is a schematic illustration of an exemplary zig-zag antimicrobial catalyst geometries;

FIG. 2 is a schematic illustration of exemplary bird's nest antimicrobial catalyst geometries; and

FIG. 3 is a scanning electron microscope image of exemplary silver flakes.

### DETAILED DESCRIPTION

The present application is more particularly described in the following description and examples that are intended to be illustrative only since numerous modifications and variations therein will be apparent to those skilled in the art. As used in the specification and in the claims, the singular form “a,” “an,” and “the” may include plural referents unless the context clearly dictates otherwise. Also, as used in the specification and in the claims, the term “comprising” may include the embodiments “consisting of” and “consisting essentially of.”

The present disclosure provides a composite, comprising a catalyst capable of promoting oxidation of organic molecules (e.g., a silver flake, a silver particle, a MnO₂ material) incorporated in a carrier, for example a hydrophilic or water-based material, the catalyst configured such that it will not discolor surrounding material under the influence of oxidative conditions. Such composite finds advantage in applications where discoloration of adjacent materials or tissue is a concern. Examples of such applications include wound dressings, where discoloration of the patient's wounds and discoloration of the dressing material is otherwise problematic, anti-odor patches, inserts, or cushions, again where discoloration of adjacent material or tissue may be problematic or unappealing, anti-bacterial adhesives, topical skincare, cleaning agents, or the like.

The present invention recognizes that previous attempts to incorporate silver (as described above, the previous attempts use silver salts in an emulsion, nanocrystalline silver or small particles of silver attached to nylon fibers) all result in discoloration of adjacent material or tissue because the silver oxidizes into black silver and leaches into the adjacent material or tissue. Without being bound by theory, silver ions destroy bacteria by oxidizing the organic material of the bacteria while the ion form of silver is reduced to the elemental form, oxidizes, and forms black silver. The black form of silver may be leached out from the carrier (e.g., a hydrogel), resulting in incorporation into wound granulation tissue and results in tissue discoloration.

When using the elemental silver, usually very small silver particles (e.g., nanoparticles) are dispersed in a hydrogel. In this situation, the mechanism is very complicated. Again, without being bound by theory, the silver particles may function as a catalyst to catalyze the redox reaction between the bacteria's organic compound and oxygen from air or other oxidant such as OH radicals. Another likely mechanism is the silver nanoparticles are oxidized to ion form first. The ion form of silver will destroy the bacteria by oxidization. In both situations the silver nanoparticles undergo the significant change in the particle shape and size. This may cause the dispersed nanoparticles to agglomerate into black silver and cause the color change. The black silver will then be leached away from the hydrogel matrix and stained on or within the mammalian tissue surface.

The present invention recognizes the problems associated with these compositions and provides a carrier, for example a hydrophilic or water-based material incorporating a catalyst configured such that it will not leach into adjacent material or tissue. Antimicrobial properties are obtained by oxidation of bacteria promoted by the catalyst. However, oxidation of the bacteria does not involve direct reaction of bacteria with the catalyst, but instead the oxidant comes from air, water or hydroxyl groups that may be in the hydrophilic or water based material. The catalyst acts as a host of the reaction, but with little direct involvement of the catalyst, there is no discoloration of in the hydrophilic or water based material, and there is no leaching out of reacted catalyst to cause discoloration at adjacent sites (e.g., in a wound).

In one embodiment, the catalyst has a size sufficient such that oxidation occurs on the catalyst within the carrier and such that the catalyst does not leach out from the carrier.

One exemplary embodiment utilizes a silver flake, having an average longest dimension of about 0.1 to about 20 micrometers, dispersed into a hydrophilic delivery system, such as a hydrogel or hydrophilic foam, as a wound dressing. The resulting composite advantageously has broad anti-bacterial properties, but will not deposit silver into surrounding material or tissue such that the material or tissue will be discolored (such discoloration of tissue is known as argyria). The physical construct of the silver flake keeps the silver anchored to the matrix of e.g., the hydrogel, permitting the solubilized silver to kill bacteria and be retained in the composite. The absorbent nature of the hydrophilic material matrix pulls fluid containing bacteria from the wound surface into the matrix, where contact with the bactericidal concentration of silver resides and exerts its effects at controlling wound and tissue surface micro flora. Accordingly, the exemplary composite effectively treats and prevents infection without discoloring tissue, without demonstrating negative effects on tissue fibroblast activity, and without discoloring in the matrix preparation.

While the above exemplary embodiment calls for a particle size of about 0.1 to about 20 micrometers, it should be recognized that any size that prevents the silver from leaching out of the carrier to discolor adjacent material or tissue is contemplated. In another embodiment, the antimicrobial catalyst has an average longest dimension of about 0.01 micrometers to about 100 micrometers.

In another embodiment, the catalyst has an irregular geometry, such that catalyst oxidation occurs on the surface of the catalyst and such that the catalyst does not leach out from the carrier.

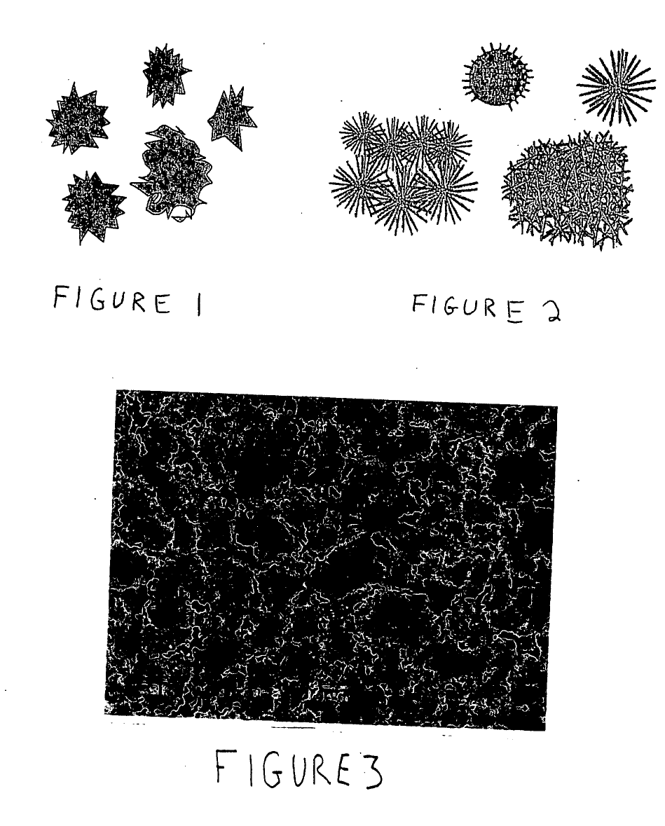

Referring to FIG. 1, a first exemplary irregular geometry is illustrated as a zig-zag path particle shape. This zig-zag shape on the catalyst surface provides high surface area and catalytic sites for bacteria oxidation and matrix anchorage. Without being bound by theory, increasing such surface area by providing for the irregular geometry provides a more stable catalyst component as well as increases catalytic-related oxidation, which will occur on the surface of the catalyst. The bacterium killing process thus will not cause substantial chemical or physical changes to the catalyst, and the catalyst will not be leached out from the composite matrix. Thus, no discoloration can take place.

Referring now to FIG. 2, a second exemplary irregular geometry is illustrated as a high surface area bird's nest shaped superstructure shape. This bird's nest shape on the catalyst surface provides high surface area and catalytic sites for bacteria oxidation and matrix anchorage. Without being bound by theory, increasing such surface area by providing for the irregular geometry provides a more stable catalyst component as well as increases catalytic-related oxidation, which will occur on the surface of the catalyst. The bacterium killing process thus will not cause substantial chemical or physical changes to the catalyst, and the catalyst will not be leached out from the composite matrix. Thus, no discoloration can take place.

In one exemplary embodiment, the product is made of a mixture of hydrogel with specifically designed particle surfaces, including flaky materials that have a zig-zag path shape (e.g., silver flakes having average longest dimensions of about 2 to about 10 micrometers) and interwoven bird's nest superstructure shapes having individual nanometer sized fibers, e.g., MnO₂ nanofibers. In another embodiment the catalyst is a metallic material. The metallic material may be a metallic oxide, intermetallic oxide, or rare earth oxide, or a combination of oxides and doped oxides. In one embodiment, the oxide may be an oxide of an element selected from a group of iron, manganese, cobalt, aluminum, zirconium, or a combination comprising at least one of the foregoing. The particles may be mixed with the hydrogel and applied onto a substrate. The composition can then be crosslinked, die-cut, packaged, and sterilized.

In another exemplary embodiment, an antibacterial sheet aqueous hydrogel wound dressing is provided, comprising about 10% polyethylene oxide with 0.05% to 0.15% silver powder (Inframat Corporation #47MR-01C), 0.1 % to 0.5% silver flakes (Inframat Corporation #47MR-12F). In an exemplary manufacturing process, the hydrogel/silver formulation is extruded between two layers of polyethylene film with a polymer net material acting as a scrim to control the gel as it expands as the result of fluid absorption during use.

Once the hydrogel-forming polymer and water are mixed, the mixture is crosslinked to form the hydrogel in sheet rollstock form. The crosslinking may be accomplished using any convenient energy source (such as an electron beam energy source). Depending on the hydrogel-forming polymer used, the ratio of the hydrogel-forming polymer and water, and the amount of antimicrobial agent and/or additional additives, the amount of energy needed may be calculated to cause crosslinking.

The crosslinked hydrogel sheet rollstock is then subjected to a die cutting procedure and packaged in standard polymer packaging which will permit sterilization via ethylene oxide gas, gamma irradiation, or electron-beam irradiation techniques.

The above-described method does not require any chemical additive to effect the crosslinking. As such, the method is more efficient and/or cost-effective as there is no need for a chemical additive to enhance crosslinking as with prior art methods.

A variety of carriers are useful. The carrier is capable of retaining the antimicrobial catalyst through covalent attachment or physical adsorption, without significantly adversely affecting antimicrobial catalyst activity. Since the carrier both supports the antimicrobial catalyst and allows access to the antimicrobial catalyst, solid, porous supports may be used, for example high surface area supports such as foams (organic or inorganic), fabrics (woven and non-woven), aerogels, and the like. In this embodiment, the antimicrobial catalyst is adsorbed or covalently bound to the surface of the support. In use, such solid, porous supports are immersed in an aqueous medium to allow access to the antimicrobial catalyst.

In one embodiment, the antimicrobial catalyst (e.g., silver flake) is incorporated into a hydrophilic pre-polymer with ultimate processing into an open or closed cell foam matrix.

In another embodiment the carrier is a hydrogel, wherein the antimicrobial catalyst is suspended in the hydrogel or associated with the polymer used to form the hydrogel. As is known in the art, a hydrogel is a network of one or more high molecular weight, usually biocompatible polymers, which can swell in water to form a gel-like material. Many different types of hydrogels are available, and selection of a particular hydrogel will depend on factors such as the intended use (i.e., cosmetics or wound care), ease of manufacture, cost, efficacy, and like considerations. Hydrogels are formed by combining a hydrogel-forming polymer with water and other optional additives.

Hydrogel-forming polymers usable for the carrier are not particularly limited, as long as the hydrogel provides a stable environment for the antimicrobial catalyst, and allows oxidation of organic material without discoloration. Hydrogel-forming polymers are hydrophilic or have hydrophilic units or segments, together with hydrophobic units or segments. Exemplary hydrophilic units (e.g., blocks) may be formed by the polymerization of monomers such as N-vinyl pyrrolidone, vinyl pyridine, acrylamide, methacrylamide, N-methyl acrylamide, hydroxyethyl methacrylate, hydroxyethyl acrylate, hydroxymethyl methacrylate, hydroxymethyl acrylate, methacrylic acid, acrylic acid, maleic anhydride, vinyl sulfonic acid, styrenesulfonic acid, lactic acid, glycolic acid, ethylene glycol, vinyl alcohol, N,N-dimethylaminoethyl methacrylate, N,N-diethylaminoethyl methacrylate, N,N-dimethylaminopropyl acrylamide, and the like.

Hydrophobic units (e.g., blocks) may be provided by the polymerization of monomers such as ethyl acrylate, methyl methacrylate, and glycidyl methacrylate; N-substituted alkyl methacrylamide derivatives such as N-n-butyl methacrylamide; vinyl chloride, acrylonitrile, styrene, vinyl acetate, and the like. The relative ratio of hydrophilic portions to hydrophobic portions will depend on the desired properties of the hydrogel.

Specific examples of hydrogel-forming polymers include polyalkylene-oxide block copolymers, for example block copolymers comprising polypropylene oxide blocks and polyethylene oxide blocks; etherified (or ether group-containing) celluloses such as methyl cellulose, carboxymethylcellulose, and hydroxypropyl cellulose; chitosan derivatives; collagen; laminin; alginates; poly(glycolic-co-L-lactic acid); polyvinyl alcohol; polyvinyl pyrrolidone; polyethylene oxide; certain polyurethanes; alkali metal salts of polyacrylic acid; polyacrylamides; ethylene-maleic anhydride copolymers; polyvinyl ethers; poly(gamma-glutamic acid); graft copolymers of starch and acrylic acid starch and saponified acrylonitrile, starch and saponified ethyl acrylate, and acrylate-vinyl acetate copolymers saponified; copolymers of maleic anhydride and alkyl vinylethers; saponified starch graft copolymers of acrylonitrile, acrylate esters, vinyl acetate, and starch graft copolymers of acrylic acid, methylacrylic acid, and maleic acid; and poly(hydroxyethyl methacrylate). Water-absorbing natural starches and gums may also be used. Some of the foregoing and other hydrogel polymers are commercially available from many sources, including Polysciences, Inc. of Warrington, Pa. (USA); BASF Wyandotte Chemical Co., Dow Chemical Company, Absorbent Technologies, and Stockhausen. A combination of hydrogel-forming polymers may also be used.

Hydrogel-forming polymers may be crosslinked during or after swelling to modify their properties, for example to improve stability. Crosslinking agents, that is, di-, tri- or higher functionality compounds that react with the hydrogel-forming polymer may be used. Exemplary functional groups include epoxy, ethylenically unsaturated groups such as vinyl, allyl, acrylate, and methacrylate, aldehyde, ketone, amido, hydroxyl, amino, and the like. The groups may be covalently attached to a small molecule, oligomer, or polymer (e.g., polyethylene glycol, polysaccharide). Exemplary crosslinking agents include polyethylene glycol diacrylate, methylenebisacrylamide, ethoxylated glycerols, inositols, trimethylolpropanes, succinates, glutarates, glycolate/2-hydroxybutyrate and glycolate/4-hydroxyproline. Catalysts as is known in the art may initiate crosslinking.

The hydrogel may comprise a hydrogel-forming polymer that is a hydrophilic polymer. The hydrogel-forming polymer may be mixed with water in a wide range of ratios. The hydrogel-forming polymer may be mixed with water in a ratio range of from about 1 part hydrogel-forming polymer to about 33 parts of water, by weight to about 1 part hydrogel-forming polymer to about 3 parts of water.

In one embodiment, the hydrogel-forming polymer is a hydrophilic polymer. Examples of hydrophilic polymers that may be used include, but are not limited to, starch, cellulose, cellulose derivatives, polyvinyl alcohol, polyalkylene oxide, polyethylene oxide, polypropylene glycol, and other hydrophilic polymers including, but not limited to, poly(1,3-dioxolane), copolymers of polyethylene oxide or poly(1,3-dioxolane), polyvinyl pyrrolidone, polyethylene glycol, polyacrylic acid poly(2-methyl-2-oxazoline polyglycidyl trimethyl ammonium chloride, polymethylene oxide, and the like. In one embodiment, polyethylene oxide is the hydrophilic polymer that is used.

Once the hydrogel-forming polymer and water are mixed, the mixture is then crosslinked to cause the hydrogel-forming polymer to crosslink with the water to form the hydrogel of the present invention. The crosslinking is accomplished using an energy source. The mixture is subjected to this energy source. Depending on the hydrogel-forming polymer used, the ratio of the hydrogel-forming polymer and water, and the amount of antimicrobial agent and/or additional additives, the amount of energy needed to cause crosslinking may vary. In general, the amount of energy used in the present invention is any amount sufficient to cause the hydrogel-forming polymer and water to form crosslinked bonds to give the hydrogel greater molecular integrity and/or thereby creating a stable sheet of hydrogel.

The energy source used in the present invention may be any energy source capable of causing the crosslinking to occur. In one embodiment, the energy source is an electron beam, such as one generated by an electron beam accelerator. In another embodiment, the energy source is gamma radiation, UV radiation, or a chemical or thermal source. Other energy sources, such as catalyst agents, that can cause crosslinking bonds between the hydrogel-forming polymer and the water may also be used in the present invention.

The energy is supplied in an amount sufficient to cause crosslinking. As this amount may vary widely due to a variety of different factors, the exact amount of energy used in forming the hydrogel is dependent on process considerations. In one embodiment, the hydrogel is formed by placing the mixture on a substrate and supplying the energy source as the substrate is passed through the energy source. Depending on the line speed, the strength of the energy field, and/or the amount of energy needed to crosslink the hydrogel-forming polymer and water, the process parameters may vary. For example, in one embodiment, the strength of the energy field may be as low as about 0.1 milliAmperes (mA). However, if small amounts of energy are needed to crosslink the hydrogel, then the line speed may be much higher than an embodiment wherein the strength of the energy field is about 40 mA, but a large amount of energy is needed to cause crosslinking of the hydrogel-forming polymer with the water.

In addition to the hydrogel-forming polymer, water and the antimicrobial agent, the composition may also include one or more additives to assist in making the hydrogels and/or to affect the final material based upon the expected end-use for the product. For example, if the end use of the hydrogel is an article that contacts the skin, a pH adjuster may be added to reduce or increase irritation of the skin. Other additives may include an anti-fungal additive and/or another antimicrobial additive for further antimicrobial protection; preservatives; and/or a salt to increase the conductivity of the hydrogel. Alternatively, in some embodiments, it may be beneficial to decrease the rate of crosslinking. As such, a crosslinking inhibitor may be used. In other embodiments, it may be beneficial to add a crosslinking enhancer. The amounts of these additives may comprise about 0 to about 10% by weight of the hydrogel. In other embodiments, these additives may comprise about 0.5 to about 5% by weight of the hydrogel.

While the described hydrogel may be produced without an enhancer, there may be instances wherein small amounts, i.e. less than about 1% by weight, of a crosslinking enhancer may be used. The resulting hydrogel would still be substantially free from any crosslinking enhancer additive. Accordingly, as used herein, the term “substantially free of any additive for enhancing crosslinking” means a hydrogel having less than about 1% by weight of the additive.

The hydrophilic or water-based material may be applied to any substrate. If the material is a hydrogel mixture, such mixture may be placed onto a substrate prior to crosslinking or after crosslinking. The substrate may be a top liner, a bottom liner, a scrim, or a combination thereof. The material containing the catalyst may also be placed such that there are multiple layers of such material with a substrate layer between the layers. For example, in one embodiment, a hydrogel may be placed on a bottom layer, with a scrim placed on the layer of hydrogel. Then, another layer of hydrogel may be applied, followed by a top liner. In this embodiment, the scrim would become embedded within the two hydrogel layers. The top liner and/or bottom liner may be made from any suitable material, such as a polyvinyl alcohol film, a polyethylene oxide film, or the like. In certain embodiments, the catalyst (e.g., silver flake) is applied to the substrate rather than being mixed with, or in addition to mixing with, the hydrogel-forming polymer and water. As a result, the catalyst may migrate into the hydrogel, thereby providing the ability to promote oxidation of organic molecules to the hydrogel. If multiple substrates are used in a particular embodiment, one or more of the substrates may have the catalyst applied thereto.

While the hydrogel may be applied to a substrate including a top sheet liner and/or a bottom sheet liner, the hydrogel in many embodiments includes a scrim for supporting the hydrogel. The scrim may be any material capable of providing support whether the scrim is located on an external surface of the hydrogel or embedded within the hydrogel. The scrim is selected such that it may be cut, sized or otherwise manipulated such that the hydrogel may include the scrim when used in the final end application. The scrim may be a mesh, a foam, a film, a woven material, and/or a non-woven material. In one embodiment, the scrim is a high-density expanded polyethylene web.

The composite described herein may be used in any application wherein it is advantageous to have antimicrobial properties, or where oxidation of organic molecules, such as bacteria, is desired. Particular areas of application of antimicrobial agents and compositions are, for example, cosmetics, disinfectants, sanitizers, hospital and medical uses, bandages and wound dressings (e.g., for exudating wounds, wound packing, blister treatment, burn care, post surgical dressings), anti-odor patches, inserts or cushioning, topical skin care, disposable diapers, and feminine care articles. The present composition may also be useful for production of drug eluting implants or drug delivery devices that can deliver drugs at specific sites with a specific delivery rate and at the same time killing bacteria resulting from injuries or medical procedures.

This disclosure is further illustrated by the following non-limiting examples:

### EXAMPLE 1

Referring now to FIG. 3, a scanning electron microscope image (with a dimension bar of 10 micrometers) illustrates exemplary silver flakes. The silver flakes have an irregular geometry with dimensions of about 8 to about 10 micrometers and about 2 to about 4 micrometers. The silver flakes were dispersed into a hydrogel, and the composite was crosslinked using an electron-beam, die cut and packaged. After 90 days in the hydrogel matrix, no discoloration of the silver within the hydrogel dressing was evident. Antibacterial study on seed plate exposure to the dressing indicated gram positive and gram negative contact inhibition with no leaching of the dressing components into the media.

### EXAMPLE 2

Silver flakes having an irregular geometry with average longest dimensions of about 0.1 to about 0.5 micrometers were dispersed into a hydrogel, and the composite was crosslinked using an electron-beam, die cut, and packaged. After 90 days in the hydrogel matrix, no discoloration of the silver within the hydrogel dressing was evident. Antibacterial study on seed plate exposure to the dressing indicated gram positive and gram negative contact inhibition with no leaching of the dressing components into the media.

The foregoing is provided for purposes of illustrating, explaining, and describing embodiments of this invention. Modifications and adaptations to these embodiments will be apparent to those skilled in the art and may be made without departing from the scope or spirit of this invention.

1. A method of making a hydrogel having antimicrobial properties, the method comprising:
mixing a hydrogel-forming polymer with water; and
applying an energy source to crosslink the mixture of hydrogel-forming polymer and water to form the hydrogel;
wherein the hydrogel further includes a silver antimicrobial agent that does not discolor.

2. The method of claim 1, wherein the hydrogel-forming polymer is a hydrophilic polymer.

3. The method of claim 2, wherein the hydrophilic polymer is selected from starch, cellulose, cellulose derivatives, polyvinyl alcohol, polyalkylene oxide, polyethylene oxide, polypropylene glycol, poly(1,3-dioxolane), copolymers of polyethylene oxide, copolymers of poly(1,3-dioxolane), polyvinyl pyrrolidone, polyethylene glycol, polyacrylic acid, polymethylene oxide, or a combination thereof.

4. The method of claim 1, wherein the hydrogel-forming polymer is added to the water in a weight ratio ranging from about 1 part hydrogel-forming polymer to about 33 parts water to about 1 part hydrogel-forming polymer to about 3 parts water.

5. The method of claim 1, wherein the silver antimicrobial agent comprises nanoparticulate silver.

6. The method of claim 5, wherein the silver antimicrobial agent further comprises silver flakes having an average longest dimension of about 0.1 micrometers to about 20 micrometers.

7. The method of claim 1, wherein the silver antimicrobial agent is added to the hydrogel in an amount of from about 0.1 to about 10% by weight.

8. The method of claim 7, wherein the silver antimicrobial agent is added to the hydrogel in an amount of from about 0.1 to about 3% by weight.

9. The method of claim 1, wherein the silver antimicrobial agent is mixed with the hydrogel-forming polymer and the water prior to application of the energy source.

10. The method of claim 1, wherein the mixture of the hydrogel-forming polymer and the water is applied to a substrate prior to application of the energy source.

11. The method of claim 10, wherein the substrate is selected from a top sheet, a bottom sheet, a scrim, or a combination thereof.

12. The method of claim 11, wherein the substrate comprises a scrim selected from a mesh, a foam, a film, a woven material, a non-woven material, or a combination thereof.

13. The method of claim 12, wherein the scrim comprises a high-density expanded polyethylene web.

14. The method of claim 1, further comprising an additive selected from an anti-fungal additive, an antimicrobial additive, a salt, a preservative, a pH adjuster, a crosslinking inhibitor, or a combination thereof.

15. The method of claim 1, wherein the energy source is selected from an electron beam, gamma radiation, or a combination thereof.

16. The method of claim 15, wherein the energy source comprises a linear accelerator electron beam.

17. A hydrogel having antimicrobial properties made according to the process of claim 1.

18. A personal care product having antimicrobial properties comprising the hydrogel of claim 17.

19. The personal care product of claim 18, wherein the personal care product is selected from a bandage, a cosmetic, a topical skin treatment, a wound dressing, a diaper, or a feminine care article.

20. The method of claim 1, wherein the hydrogel is substantially free of any additive for enhancing crosslinking.

21. A composite, comprising a carrier and particulate silver, wherein the composite does not discolor when exposed to aqueous conditions for at least about 24 hours.

22. The composite of claim 21, wherein the composite is antimicrobial.

23. The composite of claim 21, wherein the composite does not discolor surrounding material when exposed to oxidative conditions.

24. The composite of claim 21, wherein discoloration does not occur when exposed to aqueous conditions for at least about 2 days.

25. The composite of claim 21, wherein discoloration does not occur when exposed to aqueous conditions for at least about 5 days.

26. The composite of claim 21, wherein discoloration does not occur when exposed to aqueous conditions for at least about 7 days.

27. The composite of claim 21, wherein discoloration does not occur when exposed to aqueous conditions for at least about 14 days.

28. The composite of claim 21, wherein the carrier comprises water.

29. The composite of claim 24, wherein the carrier is a hydrogel.

30. The composite of claim 21, wherein the particulate silver comprises nanoparticulate silver.

31. The composite of claim 21, wherein the composite and an environment adjacent the composite do not discolor when the composite and the environment adjacent the composite is exposed to water for at least 24 hours.

32. The composite of claim 21, wherein the composite and the environment adjacent the composite do not discolor when the composite and the environment adjacent the composite is exposed to water for at least 24 hours under oxidative conditions.

33. An article that does not discolor when exposed to an aqueous envrionment for at least 24 hours, comprising the composite of claim 21.

34. A personal care product having antimicrobial properties comprising the composite of claim 21.

35. The personal care product of claim 31, wherein the personal care product is selected from a bandage, a cosmetic, a topical skin treatment, a wound dressing, a diaper, or a feminine care article.
